(19) United States
(12) Patent Application Publication
(10) Pub. No.: US 2017/0166853 A1
(43) Pub. Date: Jun. 15, 2017

(54) DIVIDABLE SURFACES FOR CELL CULTURING

(71) Applicant: Kristian EKEROTH, Huddinge (SE)

(72) Inventor: Kristian EKEROTH, Huddinge (SE)

(21) Appl. No.: 15/118,236

(22) PCT Filed: Feb. 11, 2015
(86) PCT No.: PCT/EP2015/052874

§ 371 Date: Aug. 11, 2016

Related U.S. Application Data
(60) Provisional application No. 61/938,929, filed on Feb. 12, 2014.

(51) Int. Cl. C12M 1/12; C12M 1/42; C12N 5/00; C12M 1/00; C12M 1/32

(52) U.S. Cl. CPC C12M 25/04; C12M 23/38; C12M 23/34; C12M 23/20; C12M 23/12; C12M 25/06; C12N 5/0062; C12M 35/08; C12N 5/00; C12N 2513/00; C12N 2533/50; C12N 2539/10; C12N 2502/30

(57) ABSTRACT

The current invention discloses new dividable surfaces (30) having plugs (21) and holes (11), and methods for culturing and passaging cells using said dividable surfaces (30). In particular a cell culture assembly (1) comprises a top component (10) with through holes (11) in a fixed pattern and a bottom component (20) with matching plugs (21) distributed in the fixed pattern and arranged to be aligned with the through holes (11). Walls (12) of the through holes (11) and/or walls (22) of the plugs (21) are fully or partially angled or sloped.

### BACKGROUND OF THE INVENTION

Field of the Invention

The present invention relates generally to the field of cell culturing. More specifically the invention relates to dividable surfaces for cell culturing and cell passaging.

Description of the Related Art

Cell culture is the complex process by which cells are grown under controlled conditions, generally outside of their natural environment. In practice, the term “cell culture” now refers to the culturing of cells derived from multi-cellular eukaryotes, especially animal cells. However, there are also cultures of plants, fungi, insects and microbes, including viruses, bacteria and protists. Cells are grown and maintained at an appropriate temperature and gas mixture (typically, 37° C., 5% CO₂ for mammalian cells) in a cell incubator. Culture conditions vary widely for each cell type, and variation of conditions for a particular cell type can result in different phenotypes. Aside from temperature and gas mixture, the most commonly varied factor in culture systems is the cell growth medium.

Plating density (number of cells per volume of culture medium) plays a critical role for some cell types. Cells can be grown either in suspension or as adherent cultures. Some cells naturally live in suspension, without being attached to a surface, such as cells that exist in the bloodstream. There are also cell lines that have been modified to be able to survive in suspension cultures so they can be grown to a higher density than adherent conditions would allow. Adherent cells require a surface, such as tissue culture plastic or microcarrier, which may be coated with extracellular matrix components (such as collagen and laminin) to increase adhesion properties and provide other signals needed for growth and differentiation. Most cells derived from solid tissues are adherent. Another type of adherent culture is organotypic culture, which involves growing cells in a three-dimensional (3D) environment as opposed to two-dimensional (2D) culture dishes. This 3D culture system is biochemically and physiologically more similar to in vivo tissue, but is technically challenging to maintain because of many factors (e.g. diffusion).

As cells generally continue to divide in culture, they generally grow to fill the available area or volume. This can generate several issues such as nutrient depletion in the growth media, accumulation of apoptotic/necrotic (dead) cells or cell-to-cell contact that can stimulate cell cycle arrest, causing cells to stop dividing, known as contact inhibition. To avoid these problems, one of the most common manipulations carried out on culture cells is passaging cells. Passaging (also known as subculture, expanding or splitting cells) involves transferring a small number of cells into a new vessel. Cells can be cultured for a longer time if they are split regularly, as it avoids the senescence associated with prolonged high cell density. Suspension cultures are easily passaged with a small amount of culture containing a few cells diluted in a larger volume of fresh media. For adherent cultures, cells first need to be detached; this is commonly done with a mixture of trypsin-EDTA; however, other enzyme mixes are now available for this purpose. A small number of detached cells can then be used to seed a new culture. For example, one common type of adherent cell culture is cultures of eukaryote origin, grown in culture flasks, dishes or multiwell plates in suitable media containing nutrients. Often, the bottoms of the flasks/wells are coated with a coating to make the cells attach and expand, and the nutrient media exchanged when necessary. When the cells are confluent (covering the whole culturing surface), or have reached a confluency high enough, they need to be split; i.e. transferred into new culture vessels, so that they may continue to be cultured. When splitting adherent cells the first step is to detach the cells, which is often done using an enzyme mixture of, for example trypsin-EDTA, or by mechanical methods such as pipetting, scraping and centrifugation. The detached cells are then resuspended in fresh medium, some transferred into new culture vessels, and then allowed to reattach to their growth surface.

However, the methods used today for splitting cells when culturing have several drawbacks. Enzymes, chemicals, pipetting or centrifuging have negative effects on the cells and their cultures such as stress, cleavage of surface proteins, and cell-death. Also, splitting multiwell plates is generally not practically feasible. New tools for culturing and splitting cells are therefore needed.

Patent application CA2549777 describes a modular assembly for cultivating and passaging adherent cells without the need for trypsin. The modular assembly consists of two or three interlocking stackable cell culture dishes, which allow for the separation of the growth surface into fractions. An array of flat-top pins in a lower component fit into the holes of an upper component, so that the top of the pins and the area around the holes form a continuous surface suitable for cell culture. When cells have grown to cover the surface, the components can be separated and combined with new components without cells, giving cells new surfaces to expand over.

There is a continued need for a new design to alleviate and overcome shortcomings in the prior art, allowing for efficient cell culturing and trypsin free passaging of cultured cells.

### SUMMARY OF THE INVENTION

A general purpose of the invention is to provide new dividable surfaces for cell culturing and cell passaging. The surfaces are in one embodiment consisting of two components that can be put together to form a surface for cell culture. The two components are formed like a bottom and a top component, wherein the top component has holes and the bottom component has plugs that can be fitted together to form an even surface. The cells may be cultured on this surface, and split by separating the top and bottom components, where after each component may be combined with a new bottom or top component.

Hence, an aspect of the embodiments relates to a cell culture assembly comprising a top component comprising multiple through holes distributed in a fixed pattern. The cell culture assembly also comprises a bottom component comprising multiple plugs distributed in the fixed pattern and arranged to be aligned with the multiple through holes. The multiple plugs fit in the multiple through holes. According to the embodiments, walls of the multiple through holes are fully or partially angled or sloped and/or walls of the multiple plugs are fully or partially angled or sloped.

Another aspect of the embodiments relates to a cell culture assembly comprising a top component comprising a cell culturing surface and comprising multiple through holes having at least one respective wall. The cell culture assembly also comprises a bottom component comprising multiple plugs arranged to be aligned with the multiple through holes. Each plug of the multiple plugs comprises a cell culturing surface and at least one wall. The cell culturing surface of the top component and the cell culturing surfaces of the multiple plugs form a dividable cell culturing surface when the multiple plugs are inserted in the multiple through holes. According to the embodiments, at least a bottom part of the at least one wall of the multiple through holes is sloped or angled relative to a normal of the cell culturing surface of the top component. In addition, or alternatively, at least a top part of the at least one wall of the multiple plugs is sloped or angled relative to a respective normal of the cell culturing surface of the multiple plugs.

A primary object of the invention is to provide a new method for splitting cells without the use of enzymes, such as trypsin. This may not only provide a method without the need for enzymes or chemicals, but may also provide a method for splitting which retains most cell-cell contacts and cell-surface contacts and requires no reattachment time.

Hence, a further aspect of the embodiments relates to a cell passaging method. The method comprises culturing cells on a cell culturing surface formed by a top surface of a top component of a cell culture assembly according to the embodiments and respective top surfaces of plugs of a bottom component of the cell culture assembly. The method also comprises removing the top component from the bottom component. The method further comprises performing at least one of:

attaching the top component onto a new bottom component lacking any cells on respective top surfaces of plugs of the new bottom component; and

attaching a new top component onto the bottom component. The new top component lacks any cells on a top surface of the new top component.

Yet another aspect of the embodiments relates to a cell co-culturing method comprising culturing cells on a cell culturing surface formed by a top surface of a top component of a cell culture assembly according to embodiments and respective top surfaces of plugs of a bottom component of the cell culture assembly. The method also comprises removing the top component from the bottom component. The method further comprises performing at least one of:

attaching the top component onto a new bottom component comprising cells on respective top surfaces of plugs of the new bottom component; and

attaching a new top component onto the bottom component. The new top component comprises cells on a top surface of the new top component.

The dividable surface might be used for forming perfectly sized Embryoid Bodies (EB), for achieving certain split ratios, for splitting multiwell plates, for co-culturing cells, for culturing single-clone cells in separate compartments, for splitting stem cells as lumps, and for avoiding selection of cancer prone cells when splitting.

Other objects and advantages of the present invention will become obvious to the reader. For the avoidance of doubt, the description of a feature as an ‘object’ of the invention does not necessarily imply that the object is achieved by all embodiments of the invention. Also, it is to be understood that the phraseology and terminology employed herein are for the purpose of the description and should not be regarded as limiting.

### BRIEF DESCRIPTION OF THE DRAWINGS

FIG. 1 shows the general concept of one embodiment of the invention, two dividable surfaces that can be put together, one surface having “plugs” and one surface having “holes”, wherein the plugs are made to fit in the holes to form a surface suitable for cell culturing.

FIG. 2 is a side-view illustration of the splitting process. In 2a, the two components are assembled together. In 2b, cells are added and cultured until they cover the combined surface. In 2c, the two components are separated. In 2d, the two components with cells on them are combined with new components without cells, so that the cells can expand to cover the new surface.

FIG. 3 is a side-view, close-up illustration of different plug wall and hole wall configurations. 3a shows a close-up of the plugs and holes similar to the embodiment of CA02549777. As seen, the inner walls of the holes, and the walls of the plugs, are vertical, perpendicular to the cell culture surface. 3b shows a close-up of the plugs and holes with angled sides, as proposed in the current invention. 3c shows an embodiment of the invention wherein the hole walls are angled but the plug walls are vertical all the way. In 3d, the plug has vertical walls that are angled at the uppermost part of the plug. 3e and 3f show two embodiments of the invention wherein the hole walls of the top component are both angled and vertical to form a well, which could be used for growing e.g., single cell clones.

FIG. 4 illustrates fluid flow during assembly, and additional variations of the shape of plug and hole components. 4a illustrates the flow of medium as the two components are fitted together. 4b shows an embodiment where the top component can be made thinner than the height of the plugs on the bottom component. 4c shows an embodiment of the invention with an angled plug with a vertical hole which fits around the base of the plug, which forms a gap between the plug and the hole walls, which might be used for co-culturing cells.

FIG. 5 shows an embodiment of the invention, wherein the dividable surfaces are of a size similar to a microscope glass slide. This version can be immersed in cell culture medium in a cell culture dish. A hexagonal arrangement of plugs and holes is illustrated. Larger plugs and holes situated on edges or frames, used for guiding the assembly process, are also depicted.

FIG. 6 shows two different embodiments of the invention, where inserts in holding frames are used for splitting cells in multiwell plates. 6a shows an illustration where plugs are integrated in the bottom of a cell culture well, and the hole component is applied in the form of an insert in a holding frame. In 6b, the plug component is also depicted as an insert.

FIG. 7 shows an illustration of a partial holding frame structure, holding several inserts together.

FIG. 8 shows an embodiment of the invention, wherein a top component consists of wells where the bottom of the wells have thin membranes with cone-shaped holes, and corresponding plugs are integrated in the bottom tray-like component.

FIG. 9 shows a variant similar to the device in FIG. 8, where walled compartments have been added to the bottom component, for the purpose of containing and separating culture medium from different wells. 9b shows an illustration where larger guiding plugs are placed inside the walled compartments, in proximity to the plugs used for splitting cells. 9c shows how a protruding part or element, containing a hole that aligns with a guiding plug, has been added to the outside wall of a cylindrical well.

FIG. 10 shows an embodiment of the invention, wherein small (typically 0.1-1 mm high) separating walls are added to the hole component, so that splittable micro-compartments or microwells for cell culture are formed. A fluid-permeable membrane can optionally be added on top of the separating walls, so that cells cannot leave the compartment through the top opening. In 10a, a single plug and hole is used per one microwell/microcompartment. In 10b, several plugs and holes are used per one microwell/microcompartment. Larger microwells/microcompartments may contain up to several hundreds of plugs and holes.

FIG. 11 shows an embodiment of the invention, wherein the dividable surfaces are combined with a technology such as PIPAAm to produce Embryoid Bodies (EBs). In 11a, a plug component with cells cultured on PIPAAm-coated plugs (I) is turned upside down over a non-coated plug component (III) and a hole component (II) of the type in FIG. 3f. The hole component and the non-coated plug component, marked II and III respectively, can also be combined into a single component. In 11b, all components have been assembled together, and the temperature lowered so that cells detach.

FIG. 12 shows an illustration of cutting cells to achieve higher splitting ratios. In 12a, when a hole component with a continuous layer of cells is placed on plug component 1, a laser or special cutting blades will cut and thereby deposit cells on plugs marked 1, on the next plug component cells on plugs marked 2, and so on. If a laser is used, a laser mask can block appropriate areas for the laser beam, and the beam may also be split to cut in several places simultaneously. Alternatively, the beam may be blocked by a blocking surface attached to a wheel rotating at a certain frequency, as the plate or laser assembly is slowly moved in one direction by a robot. 12b illustrates a special cutting blade that only cut cells at certain intervals.

FIG. 13 is a microscope image of HeLa cells, cultured on an uncoated polyethylene naphtalate (PEN) membrane that has been cut out with a scalpel. The membrane with cells was placed on top of a second uncoated PEN membrane without cells, in order to test if the cells would cross between the membranes. The border between the membranes can be seen running through the middle of the image. The image was taken after 8 days of incubation, showing that the cells had not crossed the gap between uncoated membranes.

FIG. 14 is a microscope image of HeLa cells cultured in the same way as in FIG. 13, the difference being that the PEN membranes were coated with Poly-D-Lysine (PDL). The image was taken after 5 days of incubation, showing that the cells could cross the gap between coated membranes.

FIG. 15 is a microscope image of MCF-7 cells cultured in the same way as in FIG. 13, on uncoated PEN membranes. The image was taken after 15 days of incubation, showing that the cells had not crossed the gap between uncoated membranes.

FIG. 16 a microscope image of MCF-7 cells cultured in the same way as in FIG. 13, on PDL-coated PEN membranes. The image was taken after 11 days of incubation, showing that the cells could cross the gap between coated membranes.

### DETAILED DESCRIPTION OF THE INVENTION AND PREFERRED EMBODIMENTS THEREOF

Throughout the drawings, the same reference numbers are used for similar or corresponding elements.

A new dividable surface for cell culture is proposed, consisting of two (or more) components that can be put together to form a surface for cell culture. The two components are formed like a bottom and a top component, wherein the top component has holes and the bottom component has plugs, that can be fitted together to form a cell culturing surface, preferably as an even surface. The cells may be cultured on this surface, and split by separating the top and bottom components, whereafter each component may be combined with a new bottom or top component. This may not only provide a method without the need for enzymes or chemicals, but may also provide a method for splitting or passaging which retains most cell-cell contacts and cell-surface contacts and requires no reattachment time.

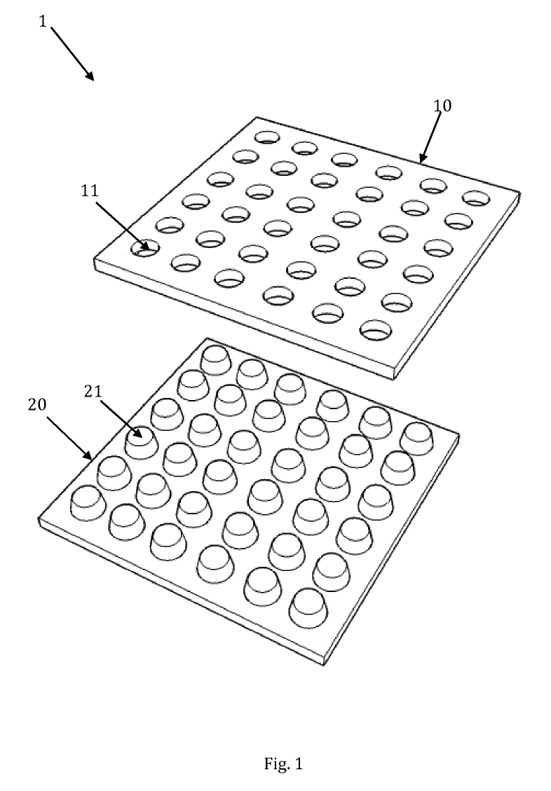

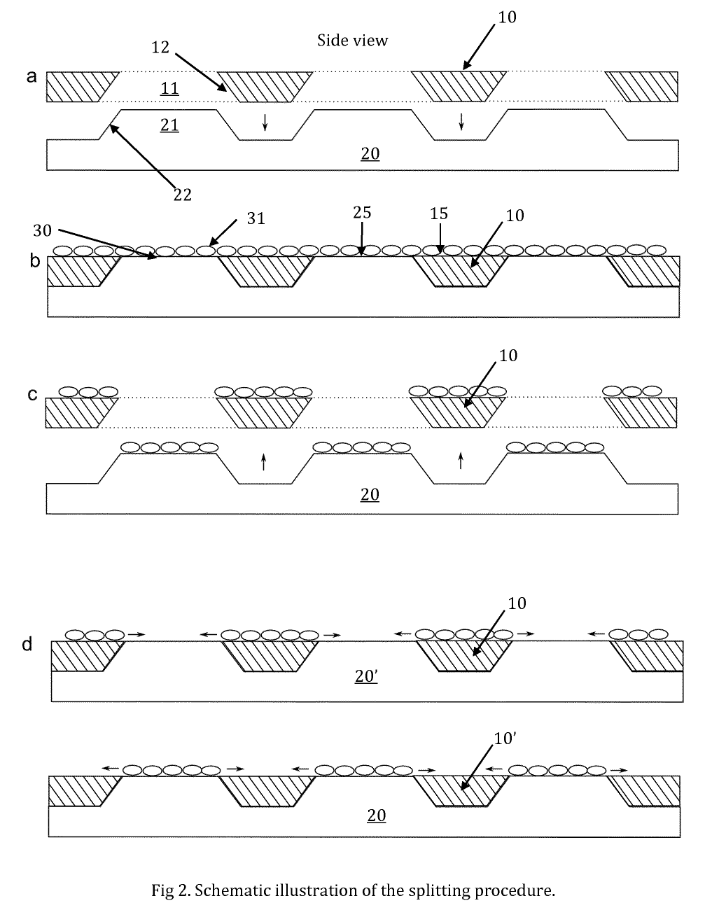

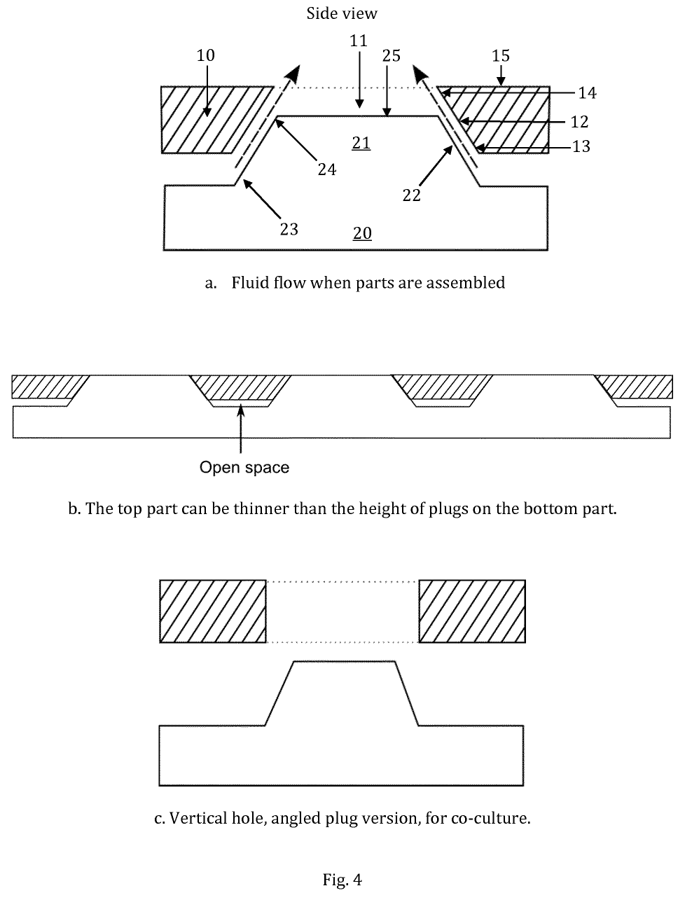

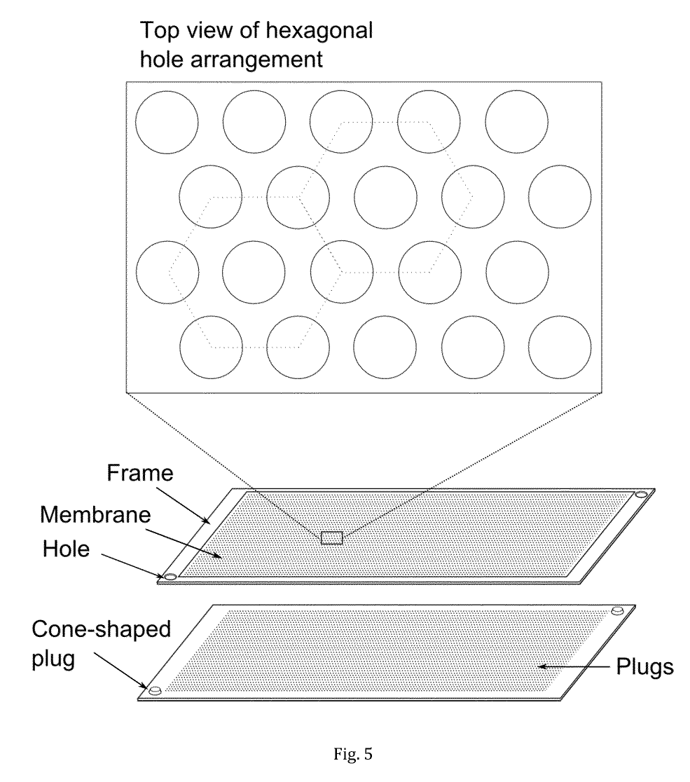

Hence, a general purpose of the invention is to provide new dividable surfaces for cell culture, in terms of a cell culture assembly 1 preferably consisting of two or more components 10, 20 that can be put together to form a surface 30 for cell culture, see FIGS. 1 and 2. The top component 10 will have holes 11, round or of other shapes, distributed in a fixed pattern, for example, a square (see FIG. 1) or hexagonal pattern (see FIG. 5). The bottom component 20 will have plugs 21 that fit in the holes 11 of the top component 10, see FIG. 1. Preferably, they form an even and essentially aligned surface 30 when put together. The shapes of the plugs 21 and holes 11 of the invention are characterized by the property or characteristic that the hole walls 12, the plug walls 22, or both the hole walls 12 and the plug walls 22 are angled, fully or partially, or sloped. In particular, for the holes 11 or plugs 21 that have angled walls 12, 22, the base 13, 23 of the hole 11 or plug 21, see FIG. 4a, is wider than the top 14, 24, which renders a space between the hole and plug walls 12, 22 during the assembly process that, when top and bottom components 10, 20 are fitted together, either is closed at the top (upper edge), at the bottom, or all the way. When the intermediate spaces are closed, the plugs 21 will be centered in the holes 11.

For the sake of clarity, the terms “plug or plugs” 21 and “pin or pins” are used interchangeable herein referring to the protrusion of the bottom component 20 of the dividable surfaces 30 and the cell culture assembly 1, to fit in the “holes” 11 of the top component 10 of the dividable surfaces 30 and the cell culture assembly 1.

The holes 11 of the top component 10 are through holes, i.e. extend through the complete thickness of the top component 10 from an upper or top main surface 15 to the opposite lower or bottom surface of the top component 10.

For the sake of clarity the terms “part or parts” and “component or components” 10, 20 are used interchangeably herein. Furthermore, the expressions “bottom component” and “plug component” or “component with plugs” are used interchangeably herein. Correspondingly, the expressions “top component” and “hole component” or “component with holes” are used interchangeably herein. The two components may be implemented as two membranes as an illustrative but non-limiting example.

An object of the invention is a new method for splitting (passaging) cultured cells 31, by using the dividable surfaces 30 of the cell culture assembly 1. When cells 31 have spread over both components 10, 20 of the dividable surfaces 30, the cells 31 can be split by separating the two components 10, 20. The top component 10 can then be combined with a new bottom component 20′, and the bottom component 20 with a new top component 10′. The procedure is illustrated in FIG. 2. This requires no enzymes, chemicals, pipetting or centrifuging, commonly used in standard splitting/passaging techniques. Most cell-cell-contacts and cell-surface-contacts are preserved, thus there is no reattachment time, and most probably less stress and cell death. It is especially important not to stress cells 31 when running a long assay, even if the cells 31 need to be split during that assay. When cells 31 are detached from their environment it affects a lot of signalling pathways, which might compromise the result of the assay. The use of the dividable surfaces 30 of the invention will facilitate the splitting of sensitive cell types, such as primary cells. The invention might also be particularly useful in the field of toxicology, where it is especially important not to stress the cells 31.

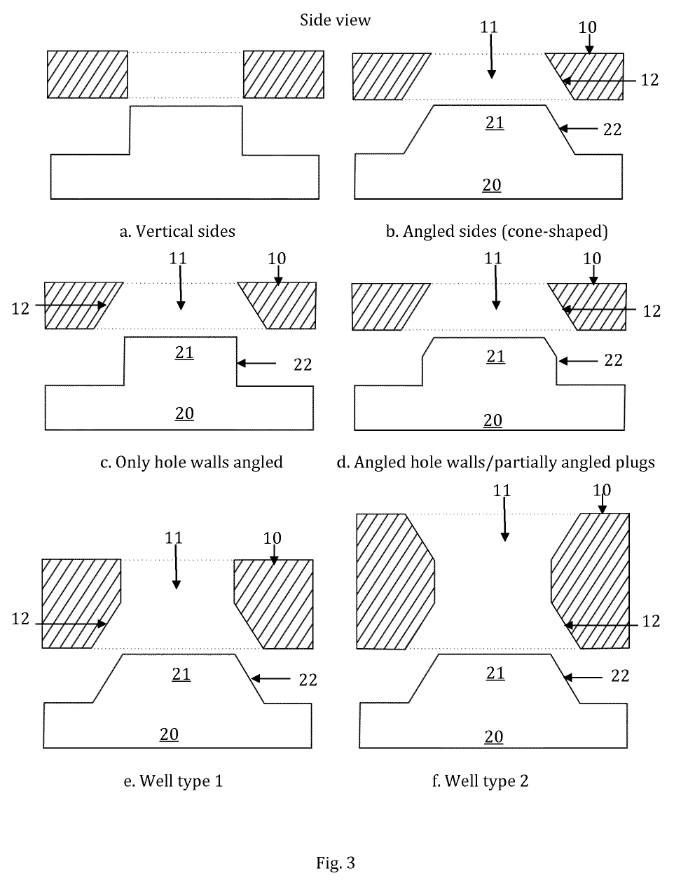

In CA2549777 mentioned in the background section, the pins and holes have the basic design of a 90 degree angle between the bottom surface and the pins, or the top surface and the holes. The inner walls of the holes, and the walls of the pins, are vertical, perpendicular to the cell culture surface. A schematic drawing of the embodiment of CA2549777 is shown in FIG. 3a. However, there are shortcomings with this design. The gap between the pin walls and the hole walls is a crucial factor. This gap cannot be too wide if cells are going to be able to cross the gap. But if it is narrow, it will increase the friction between the surfaces when the components are put together, and there is an increased risk that small misalignments block the insertion of the pin into the hole.

In CA2549777, the pins have a diameter of 5 mm. But for commercial use of the invention, it would be desirable for the pins to be much smaller, in the range of 50-1000 micrometres. Mammalian cells have diameters from around 10 micrometres to 120 micrometres (mature female oocytes). If the pins and the areas around the holes are too large compared to the cell diameters, this means that when old components are combined with new components, most cells on old components will be too far away from the edge to be able to expand over to a new component. These cells may stop dividing. If the pins and holes are smaller, this also means that their numbers will increase.

Further, in many applications it will be desirable to grow cells on much larger areas than what is available in a 35 mm dish (see table 1), so the number of pin/holes will be very large. One component can be combined, separated and re-combined with other components several times, so its shape and fit needs to be preserved with great precision, otherwise the combinations can fail. Changes in temperature, swelling due to immersion in cell culture medium, and aging of the material on the shelf are factors that could potentially cause small distortions that can lead to misalignment.

It is generally no problem if the diameter of the pin is larger than the height of the pin, but if the pin is much higher than the base is wide, so that the pin is long and slim, it increases the risk of miniscule bending causing misalignment. So a smaller diameter means shorter height, and this in turn means that the component with the holes must be thinner, since that component cannot be thicker than the height of the pin if the combined surface is going to be continuous. In CA2549777, the components with holes are solid and rigid, but when the scale decreases, the hole components would in most cases be like thin membranes attached to rigid holding frames. Such membranes are likely to have some flexibility, and this is an additional factor that can cause misalignment when using the design disclosed in CA2549777. Also, if the hole components are thin, this will reduce the area that can cause friction between hole walls and pin walls. This area can be quite large if there are many holes per area unit.

Taken together, these factors will make it difficult to reduce the scale of the holes and plugs, using the design in CA2549777, without having misalignment and friction problems. If the scale cannot be reduced sufficiently, it will take longer for cells to cover the surface, and more cells will stop dividing. This will make the device less interesting as an alternative to traditional enzymatic passaging. Another issue is what happens with cells growing on the pin component, when a hole component is replaced with a new one. Some cells that have parts sticking out over the edge of the pin may be cut or crushed by the new hole component when it is pushed down over the pins. Importantly, CA2549777 also contains no solutions for splitting multiwell plates.

According to preferred embodiments, the plugs 21 and holes 11 will probably have diameters in the range of a few hundred micrometres, and the top component 10 can be a membrane with thickness in the same range. In order to facilitate the fitting of the two components 10, 20, the sides/walls 12, 22 of the holes 11 and plugs 21 will not be vertical as in the prior art (FIG. 3a), but preferably angled as shown in FIG. 3b. However, for the purpose of splitting cells 31, it is not necessary for both the plug walls 22 and the hole walls 12 to be angled. It is possible to have vertical plugs 21, see FIG. 3c, or partially angled plugs 21, see FIG. 3d, as long as the holes 11 are angled. It is also possible to shape the top component 10 like a double membrane or component, with vertical holes on top of angled holes 11 as shown in FIG. 3e. The angled part of the hole 11 would then fit with the plug 21 and the vertical hole part would form a well. If the top component 10 is made even thicker, the top part of the holes 11 can be made angled so that the top opening is wider than the middle part of the well, see FIG. 3f. Wells such as those illustrated in FIGS. 3e and 3f could be used to grow single cell clones, perhaps distributed by an automatic system.

If both the plugs 21 and holes 11 are angled, it is possible for the top component 10 with holes 11 to be thinner than the height of the plugs 21, as illustrated in FIG. 4b. It is also possible to combine angled plugs 21 with vertical holes 11 as indicated in FIG. 4c. Then there will be a gap between the plug 21 and the hole 11, which the cells 31 cannot pass. This can be used for co-culturing cells 31, so that one type grows on the plug component 20 and another on the hole component 10. Cells can be grown together for a specific period of time and stimulate each other with, for example, soluble signalling molecules, and then be separated.

For commercial use of the invention, it would be desirable for the plugs 21 to have diameters in the range of 50-1000 micrometres. Mammalian cells have diameters from around 10 micrometres to 120 micrometres (mature female oocytes). If the plugs 21 and the areas around the holes 11 are too large compared to the cell diameters, this means that when old components 10, 20 are combined with new components 10′, 20′, most cells 31 on old components 10, 20 will be too far away from the edge to be able to expand over to a new component 10′, 20′. These cells 31 may stop dividing. If the plugs 21 and holes 11 are smaller, this also means that their numbers will increase. Table 1 below demonstrates the effect of hole/plug diameter on the number of holes/plugs, assuming that the plug area and the area around the holes are to be of equal size.

TABLE 1

| Hole | Hole | Holes per square | Holes per 35 mm |
|---|---|---|---|
| diameter (μm) | area (μm²) | mm (50% coverage) | well (962 mm²) |
| 50 | 1963 | 255 | 244971 |
| 100 | 7854 | 64 | 61243 |
| 200 | 31416 | 16 | 15311 |
| 300 | 70686 | 7 | 6805 |

In many applications it will be desirable to grow cells 31 on much larger areas than what is available in a 35 mm dish or well, so the number of plugs 21/holes 11 will be very large. One component 10, 20 can be combined, separated and re-combined with other components 10′, 20′ several times, so its shape and fit needs to be preserved with great precision, otherwise the combinations can fail. Changes in temperature, swelling due to immersion in cell culture medium, and aging of the material on the shelf are factors that could potentially cause small distortions that can lead to misalignment.

It is generally no problem if the diameter of the plug 21 is larger than the height of the plug 21, but if the plug 21 is much higher than the base 23 is wide, so that the plug 21 is long and slim, it increases the risk of miniscule bending causing misalignment. So a smaller diameter means shorter height, and this in turn means that the component 10 with the holes 11 must be thinner, since the component 10 cannot be thicker than the height of the plug 21 if the combined surface 30 is going to be continuous and even. Also, if the hole components 10 are thin, this will reduce the area that can cause friction between hole walls 12 and plug walls 22. This area can be quite large if there are many holes per area unit, as can be seen from Table 2 below.

TABLE 2
Membrane thickness = 200 micrometres

| Hole | Hole wall | Holes per one | Hole wall area per |
|---|---|---|---|
| diameter (μm) | area (μm²) | 35 mm well | 35 mm well (cm²) |
| 50 | 31416 | 244971 | 77 |
| 100 | 62832 | 61243 | 38 |
| 200 | 125664 | 15311 | 19 |
| 300 | 188496 | 6805 | 13 |
| 400 | 251327 | 3828 | 10 |

A design that addresses the design requirements discussed above is thus depicted in FIG. 3b. Here, the walls 12 of the holes 11, and the walls 22 of the plugs 21, are angled/sloped so that the holes 11 resemble cut-off funnels, and the plugs 21 resemble cut-off cones. In a preferred embodiment, the lower opening 13 of the hole 11 is wider than the top part 24 of the plug 21, while the upper opening 14 of the hole 11 fits close to the top part 24 of the plug 21. This produces a “self-aligning” effect, which makes the fitting together of the components 10, 20 much easier, and less vulnerable to small defects, since the plugs 21 align with the holes 11 while sliding in. Also, the gap between the hole walls 12 and the plug walls 22 in FIG. 3b is “self-closing”, so it is no longer necessary to find a compromise between a small gap, which facilitates cell migration, and a larger gap, which facilitates assembling the two components 10, 20, as would be necessary when using a configuration as depicted in FIG. 3a. Small amounts of flexibility in the hole component 10 is no longer a problem, as the area between the holes 11 can be allowed to stretch somewhat when the plugs 21 are inserted. Larger-scale versions of the funnel/cone system, placed on the outer frames of the components 10, 20 as in FIG. 5, can be expected to be sufficient to guide the combination of the components 10, 20. This means that both automatic and manual handling should be feasible.

A further advantage of the designs of the invention is the flow of cell culture medium when components 10, 20 are put together. In the design of the prior art (shown in FIG. 3a), the gap between the hole wall may be too tight for the medium to flow through. Rather, the medium will be pushed out at the sides of the components, or holes will need to be made in either component. But with the angled design, the medium can flow up through the closing opening, as in FIG. 4a. Cell parts that hang out over the sides can then be pushed upwards by the flow, so that the risk of cutting or crushing is decreased. Also, if loose cell parts end up on top of the new component, this can facilitate migration. If the plugs 21 are not fully angled/sloped, as in FIGS. 3c and 3d, the fluid flow may be different. When using an automated system, empirical testing can deduce the configuration and lowering speed that optimizes the fluid flow.

Other modifications of the concept of the invention could also be possible. The opposite configuration, with angled plugs 21 and straight holes 11 as shown in FIG. 4c, produces a larger gap that is particularly useful for co-culture of cells 31, when one does not want two different populations of cells 31 to be in direct contact with each other. One might try to achieve this with straight pins, straight holes and a larger gap. However, that solution does not guarantee that the gap remains uniform. If the hole component gets nudged very slightly in one direction, or flexibility in the component makes it difficult to keep all distances the same, some or all hole/pin-combinations may end up with the pin off-centred. The solution with angled plugs 21 makes sure that the gap is always the same, since the wider base functions to keep the holes 11 perfectly aligned.

The cell culture assembly 1, including its top and bottom components 10, 20 can be produced by e.g. casting, injection molding, (rapid) 3D printing (see Schubert C et al 2014 for an overview of 3D printing), or hot embossing, where a mold is pressed into a piece of heated polymer membrane. A cell culture assembly with the design features of this invention and as illustrated in the figures can be designed and then printed using a 3D printer such as Objet24, Stratasys, Eden Prairie, Minn.). Alternatively, commercially available 3D printers based on stereolithography (SLA) technology can produce features with a precision down to 0.1 micrometers (OWL MC-1 and MC-2, Old World Labs, Norfolk, Va., US.

Non-limiting examples of suitable cell culture assembly materials could be polyethylene naphthalate (PEN), polypropylene, polystyrene, polyethylene, polycarbonate, PMMA, OSTE polymers (for OSTE polymers see Errando Herranz C et al 2013), but other materials not mentioned here could also be used. The material should preferably be suitable for coating.

In order to facilitate cell migration between the components 10, 20, and to minimize the risk that cells 31 growing on plugs 21 will be pulled off from the surface 25 when the components 10, 20 are separated, it can be advantageous to coat the cell culture surface 30 with substances that promote cell adhesion and migration. Therefore, the material for the cell culture assembly 1, including the components 10, 20, should be suited for coating with substances such as Poly-D-lysine (PDL), Poly-L-Lysine (PLL), laminins, Polyethylenimine (PEI) (for PEI see Vancha A R et al 2004), or different types of collagen.

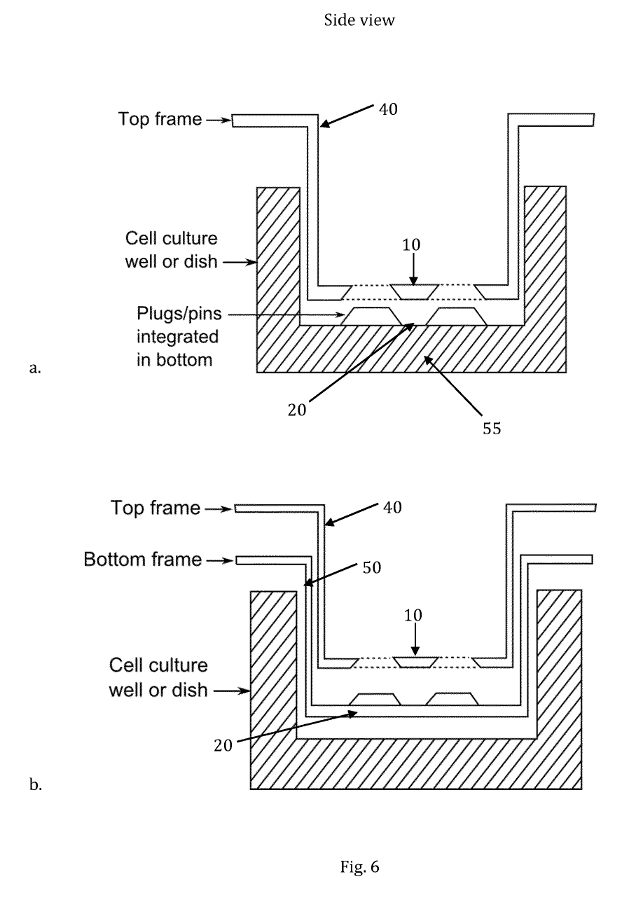

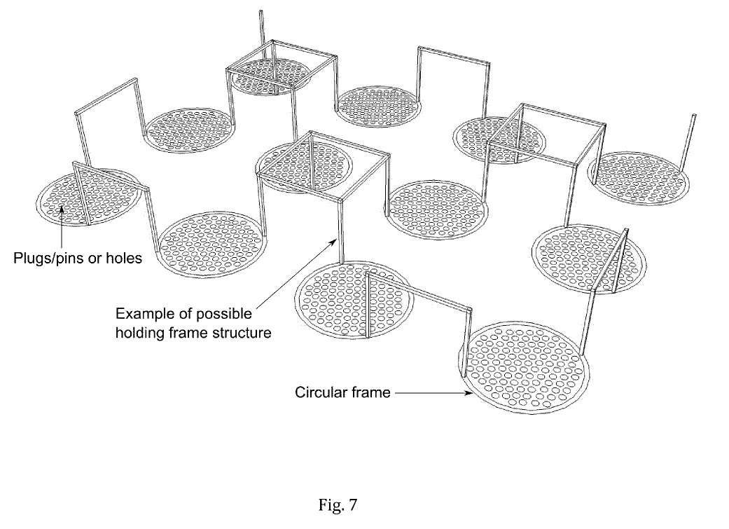

Components 10, 20 in the form of thin membranes can be attached to rigid frames stretching around their circumference, as in FIG. 5. Such a cell culture assembly or device 1 could simply be placed in a dish with cell culture medium. But the bottom component 20 can also be integrated into the bottom of cell culture dishes 55 or wells in multiwell plates 55, see FIG. 6a. A holding frame 40, 50 could then hold several thin membranes 10, 20 together. For example, bottom components 20 could be integrated in the bottom of all the wells in a 96-well plate 55, see FIG. 6a. A holding frame 40 (partly illustrated in FIG. 7), holding 96 top membranes 10, could then be inserted into the plate 55. Alternatively, there could be one holding frame 50 for the bottom membranes 20, and one frame 40 for the top membranes 10, and these would be combined to make up a cell culture insert in each well, see FIG. 6b. This way, it would be possible to easily split multi-well plates by simply exchanging holding frames 40, 50. This would be desirable especially during long experiments, for example, experiment lasting more than 24, 48 or 96 hours and where the cells may be close to reaching 80%, 90% or 100% confluence at the start of the experiment, where cell death would otherwise be a problem. When both top and bottom membranes 10, 20 are in holding frames 40, 50 as shown in FIG. 6b, it would also be possible to replace medium by simply moving both frames 40, 50 to a new plate with fresh medium. In ordinary wells, pipetting fresh medium can sometimes cause severe detachment of cells.

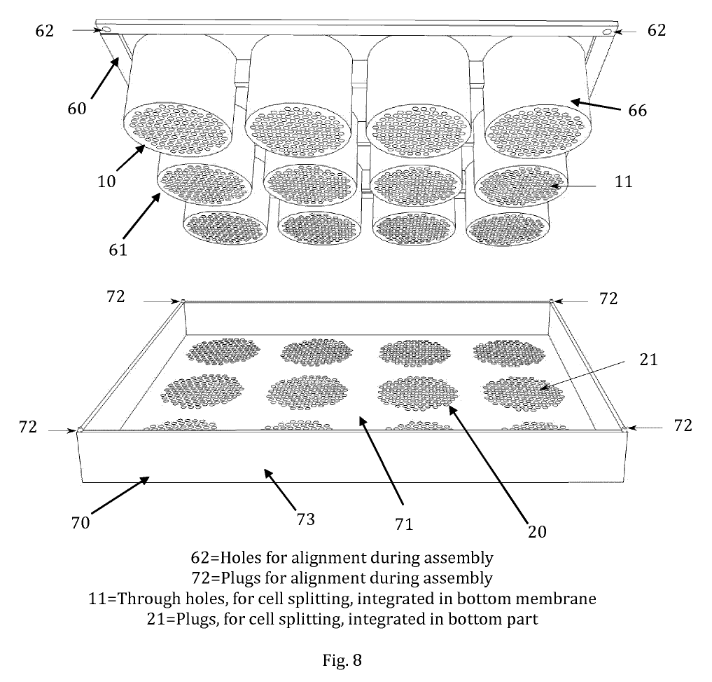

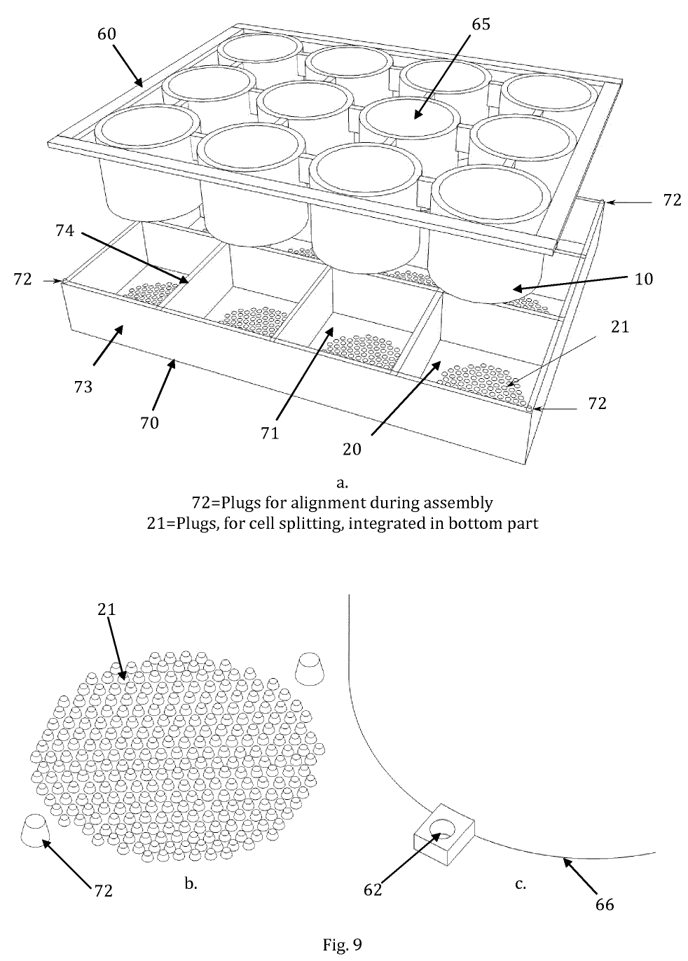

An alternative solution for splitting cells in the multiwell format is a device where the top component 10 consists of wells 65 similar to the wells in a standard multiwell plate, but where the bottoms 61 of the wells 65 are replaced with thin membranes with holes 11, see FIGS. 8 and 9. This is combined with a second tray-like component, where plugs 21 are integrated in the bottom 71 of the tray 70, as shown in FIG. 8. The membranes and the plugs 21 form a continuous surface 30 for cell culture when the two components 10, 20 are assembled together. As in other solutions, cells are passaged by taking the components 10, 20 apart, and combining them with new components 10′, 20′. In FIG. 9, separating walls 74 have been added to the bottom tray 70. This is useful if one wants to have the positive effects of fluid flow during assembly, and therefore does not want to remove the culture medium before splitting. With the separating walls 74, culture medium from different wells 65 will not be mixed together.

The technology will be well suited to automation, and the high precision in robot movements should be an advantage. Automatic systems can be fitted with cameras and software that automatically decides when the cells are confluent enough for splitting. The holding frames 60 or top components 10 may have a few larger (such as a few mm) cone-shaped plugs 72 and holes 62, or similar, to guide the handling frames 60 or top components 10 into the right spot, as in FIGS. 5, 8 and 9. In some embodiments, such as the dividable multiwell plates in FIGS. 8 and 9, guiding plugs 72 can also be placed in proximity to the plugs 21 used for splitting cells, as shown in FIG. 9b. The corresponding guiding holes 62 can be integrated in the walls 66 of the wells 65, or located in an outside protrusion of the well wall 66, as in FIG. 9c.

When lifting up a frame 60 or top component 10 that has been used, it could be useful to place some kind of drip protection between the holding frame 60 and the multiwell plate or bottom tray 70, or between the top component 10 and bottom component 20 below, before moving it horizontally. This is to avoid drop contamination between wells.

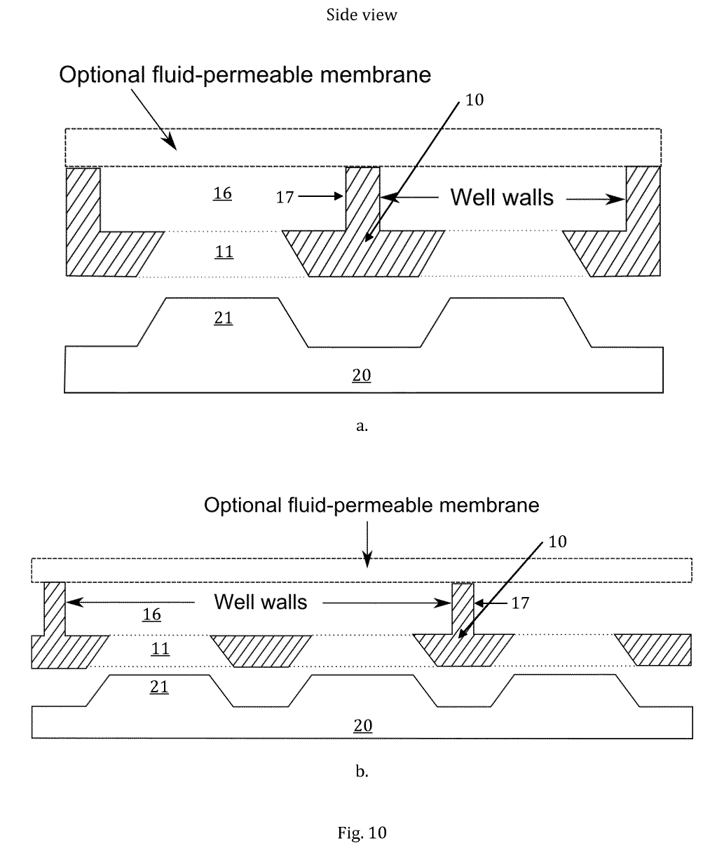

The technology can also be used to produce dividable micro-wells 16, where the micro-wells 16 or micro-compartments 16 have diameters in the range of, typically, 0.1-2 mm. This is achieved by adding separating walls 17 to top membranes 10 with holes 11, as illustrated in FIG. 10. Micro-wells have been described in the prior art, for example in US20110237445 A1, but the previously described micro-wells cannot be split. One application for micro-wells is to grow a large number of single cell clones, but if the micro-wells cannot be split, there is a limit to how long the cells can be cultured. With the dividable surfaces technology, cells can be cultured for indefinite periods of time. Also, the technology of the invention makes it easy to produce several identical arrays of cell clones through splitting, such that several different assays can be performed on all clones, even if the assays destroy the cells. For small micro-wells 16, it may be sufficient to use one single plug 21 and one single hole 11 per micro-well 16, as shown in FIG. 10a. However, a larger number of (one or more) plugs 21 and holes 11 can also be used as shown in FIG. 10b. A fluid-permeable membrane can also be applied to the top of separation walls 17 to prevent cells from escaping through the top opening as shown in FIG. 10.

The dividable surfaces of the invention might be particularly useful when culturing stem cells. Human stem cells tend to die when separated into single cells using enzymes, such as trypsin. One crucial factor is cell-cell adhesion and signalling, especially via E-cadherin. An alternative for avoiding stem cell death is to keep the cell-cell bindings intact, and let the cells stick together as lumps. Using the dividable surfaces of the invention it is possible to split stem cells as lumps, without the use of enzymes or inhibitors. There is no need to detach the cells from the surface or cutting them, if a split ratio of approximately 50/50 is acceptable. The size of the lumps may be controlled by altering the size of the pins. The cells will not end up randomly, but in a structured way after splitting since the distance between the plugs is constant. The size of cell colonies has been shown to make a difference when controlling pluripotency and grade of differentiation (see, for example, Hohenstein Elliott K A et al 2012), thus controlling size and spread might be very important for quality and reproducibility. Using dividable surfaces is also faster, needs no reattachment time, and is less complicated, an advantage for automation. Further, using the technology of the invention when passaging stem cells, there is less stress on the cells, which might render it possible to phase out different animal-derived media-components used today, media-components that potentially contain harmful substances, virus or proteins. A further aspect of the invention is to avoid the selection of cancer prone cells when splitting stem cells. Studies have shown that the risk of mutations and chromosomal alterations increases if cells are divided into single cells using enzymes, such as trypsin, compared to cells split mechanically (see, for example, Mitalipova M M et al 2005). There is a risk with using enzymes, such as trypsin, that cells less dependent on cell-cell contacts are selected, which might be a selection of cells more likely to be cancer prone. Using the method of the invention avoids using chemicals, such as enzymes, and single-cell-splitting when splitting cells, thus minimizing the risk of selecting cancer prone cells.

The need for non-enzymatic methods for splitting stem cells has prompted scientists to test stretchable surfaces as a way to expand the surface available to cells (Majd H et al 2009). This requires specialized machinery and there is a limit to how far the surfaces in question can be stretched before the cells have to be split with enzymatic methods, but it demonstrates that stem cells are able to thrive in circumstances where they are being physically pulled apart. The forces acting on stem cells when using dividable surfaces are of a similar nature.

A yet further embodiment of the invention is the combination of dividable surfaces with thermoresponsive polymers such as PIPAAm. There is an existing technology, sold under the name UpCell by Nunc/Thermo Scientific, under a license from CellSeed Inc, where cell culture surfaces are coated with a polymer, poly(N-isopropylacrylamide) (PIPAAm), in a layer that is about 15-20 nm thick. This polymer is hydrophobic above a certain temperature, typically 32 degrees C., called the Lower Critical Solution Temperature (LCST), but more hydrophilic below the LCST. The LCST can be adjusted by incorporation of other polymers. Using this technology, cells can be made to detach by simply lowering the temperature, including, but not limited to incubation for 15-40 minutes at 20-25 degrees C. According to Nunc, 5-6 minutes of incubation time can also suffice if one wants to lift the cells with forceps. This could be interesting to combine with the dividable surfaces. One application is to use a PIPAAm coating in the last splitting step, such that the cells can be detached as a sheet, which can be used in tissue engineering, by, for example, applying the sheet to a scaffold. Then the whole process, from a few cells to tissue, could be done without the use of enzymes, and without dissociating into single cells. It is also possible to grow different cells on the plug and hole components 20, 10 of the invention, let them form continuous sheets, pull them off and then stack such sheets on top of each other. The cells growing on the plugs 21 could then form “tubes” between the cells grown on the hole component 10. For example, nerve cells could form tubes between glia cells, and endothelial cells that can form veins could be embedded in muscle cells. The diameter of the pins will determine the diameter of these vertical cell structures. Examples of a method for coating a surface with temperature-sensitive PIPAAm polymer is described by Nagase et al (Nagase K et al 2009) and examples of how cell sheets can be picked up and stacked in a 3D-structure are described by Muraoka et al (Muraoka M et al 2013) and Haraguchi et al (Haraguchi Y et al 2012).

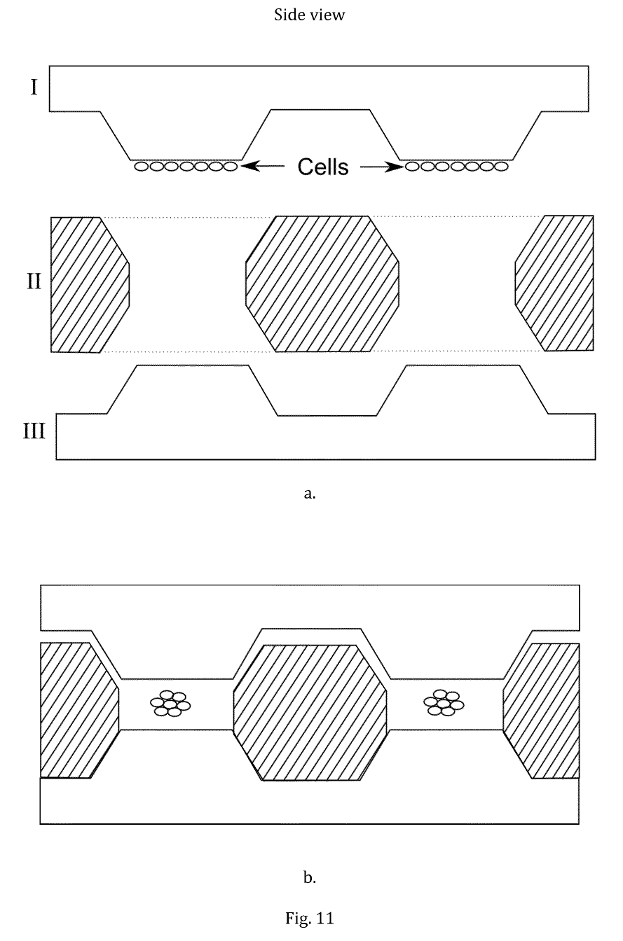

The combination with thermoresponsive polymers such as PIPAAm could also be used to easily produce perfectly-sized Embryoid Bodies (EB:s) when growing stem cells. Plugs 21 coated with PIPAAm can then be used during the final splitting stage, and the plug size could determine the number of cells that can form an EB when the cells are detached. It is also possible to isolate the EB in individual wells, as illustrated in FIG. 11. A plug component (I) with cells grown on PIPAAm-coating is in FIG. 11a turned upside down over a second, uncoated, plug component (III) and a middle well-forming component (II, also depicted in FIG. 3f). Component III is made of a material that cells do not attach to. In FIG. 11b, the three component are assembled together and the temperature has been lowered so that the cells detach, depositing them into the wells. The PIPAAm-coated part can be removed afterwards. It is also possible to have a part design where components II and III are combined into one single component.

Having EB:s of the right size is crucial for stem cell differentiation, and this would be an alternative to labour-intensive methods like “hanging drop”, and commercial products like StemCell Technologies AggreWell 400 and 800, (cell culture plates containing micro-wells with a diameter of 400 or 800 micrometres). In the AggreWell system, a suspension of cells (grown in another culture system and harvested with enzymes) is added, the plates are centrifuged to remove air bubbles, and the cells are allowed to aggregate in the wells. The technology of the invention could do the same thing as the AggreWell plates, with the difference being that the cells can be kept in the same system the entire time, from first expansion, and without the need to use enzymes in the process. The plugs 21 could have diameters similar to the micro-wells in AggreWell plates, and the sloped/angled design will make it easier to produce a system of this scale.

The combination with PIPAAm-coating could further be used to achieve higher split ratios than 50/50. In the basic variant, the split ratio is about 50/50, but higher ratios can be achieved by PIPAAm-coating the plugs 21. In the simplest variant, after the cells have grown to sufficient confluency, the top component 10 with holes 11 is replaced with a new component 10′ without cells. The cells growing on the plugs are then detached by reducing the temperature, collected by pipetting and the resulting clumps are diluted and spread over new culture surfaces, as in standard cell splitting. With this method, the size of the clumps is precisely controlled, but not the distribution pattern on the new surface.

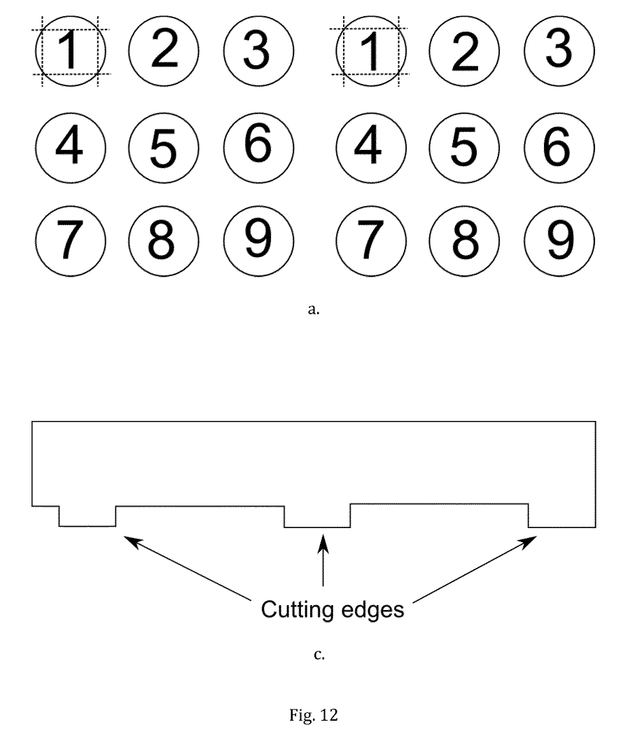

However, control over the distribution pattern can also be achieved by PIPAAm-coating the plugs 21. By reducing the temperature, the cells grown on plugs 21 will attach stronger to the other cells grown on the hole component 10 than to the plug surface 25. Then the whole sheet could be lifted up with the hole component 10, and the hole component 10 could be placed on several different plug components 20′ after one another, where some cells could be cut off (computer-controlled) and deposited on the plugs 21 in predetermined patterns, as illustrated in FIG. 12a. When the hole component 10 is placed on the first plug component, a laser or specially designed cutting blades will cut and thereby deposit cells on plugs marked 1, on the next plug component cells on plugs marked 2 will be cut, and so on. If a laser is used, a laser mask can block appropriate areas for the laser beam, and the beam may also be split to cut in several places simultaneously. Alternatively, the beam may be blocked by a blocking surface attached to a wheel rotating at a certain frequency, as the plate or laser assembly is slowly moved in one direction by a robot. Laser cutting of stem cells has been described previously (Hohenstein Elliott K A et al 2012). If cutting blades are to be used, it would be possible to use a so-called tissue chopper, similar to the McIlwain Tissue Chopper. The cutting rate for that device is 200/minute. A chopper could have several cutting blades at specified distances so that the cutting becomes faster, and the blades could be specially prepared, so that they only cut the cells at specific intervals, as illustrated in FIG. 12b.

Another embodiment of the invention is to grow cells either on the plug component 20 or the hole component 10, and have an electronic circuit on the other component, which can interact with the cells via electric signals to and from for example, cell protrusions.

An aspect of the embodiments relates to a cell culture assembly 1 comprising a top component 10 comprising multiple through holes 11 distributed in a fixed pattern. The cell culture assembly 1 also comprises a bottom component 20 comprising multiple plugs 21 distributed in the fixed pattern and arranged to be aligned with the multiple through holes 11. The multiple plugs 21 fit in the multiple through holes 11. According to the embodiments, walls 12 of the multiple through holes 11 are fully or partially angled or sloped and/or walls 22 of the multiple plugs 21 are fully or partially angled or sloped.

The fixed pattern in which the through holes 11 are arranged in the top component 10 and the plugs 21 are arranged in the bottom compartment 20 could be any defined pattern. Preferred examples include a matrix or squared pattern or a hexagonal pattern. Other patterns are though possible as long as the same pattern are used for the plugs 21 on the bottom compartment 20 as for the through holes in the top compartment 10.

The cross-sectional configuration of the plugs 21 and through holes 11 could be any defined configuration as long as the plugs 21 are able to enter and fit into the through holes 11. For instance, the plugs 21 and through holes 11 could have a circular, elliptic, quadratic, rectangular, pentagonal, hexagonal, etc. cross-sectional configuration. Preferably, the plugs 21 and through holes 11 have circular cross-sectional configuration as shown in FIG. 1.

In an embodiment, a respective base 13 of the multiple through holes 11 is wider than a respective top 14 of the multiple through holes 11 and/or a respective base 23 of the multiple plugs 21 is wider than a respective top 24 of the multiple plugs 21.

In an embodiment, the multiple through holes 11 have a round shape and the walls 12 of the multiple through holes 11 are beveled (see FIGS. 3b, 3c, 3d) or chamfered (see FIG. 3e, 30 to have a larger diameter at the respective base 13 of the multiple through holes 11 as compared to at the respective top 14 of the multiple through holes 11. In addition, or alternatively, the multiple plugs 21 are in the form of a respective chamfered circular cylinder (see FIG. 3d) or truncated circular cone (see FIGS. 3b, 3c, 3e, 30 having a larger diameter at the respective base 23 of the multiple plugs 21 as compared to at the respective top 24 of the multiple plugs 21.

In a particular embodiment, the multiple through holes 11 are multiple truncated circular funnels and the multiple plugs 21 are multiple truncated circular cones.

In an embodiment, a diameter of the respective base 13 of the multiple through holes 11 is larger than a diameter of the respective top 24 of the multiple plugs 21. In this embodiment, a diameter of the respective top 14 of the multiple through holes 11 is substantially the same as the diameter of the respective top 24 of the multiple plugs 21. Hence, any gap between a plug 21 and a through hole 11 when the plug 21 has entered the through hole 11 is very small.

In an embodiment, see FIG. 4c, the walls 12 of the multiple through holes 11 are vertical and the walls 22 of the multiple plugs 21 are fully or partially angled or sloped thereby forming a gap between a respective top 24 of the multiple plugs 21 and a respective top 14 of the walls 12 of the multiple through holes 11. Such a gap can be useful to prevent cells present on the top surface 25 of the plug 21 to enter the top surface 15 of the top component 10. This allows co-culturing of different cell types, for instance with one cell type present on the top surface 25 of the plugs 21 and another cell type present on the top surface 15 of the top component 10.

In an embodiment, top surfaces 25 of the multiple plugs 21 and a top surface 15 of the top component 10 collectively form a dividable cell culturing surface 30.

In a particular embodiment, the top surfaces 25 of the multiple plugs 21 and the top surface 15 of the top component 10 form an essentially aligned and even dividable cell culturing surface 30.

In this embodiment, the height of the plugs 21 is preferably essentially the same as the thickness of the top component 10. This means that when the plugs 21 have fully entered the through holes 11 the top surfaces 25 of the plugs 21 and the top surface 15 of the top component 10 are aligned forming an even cell culturing surface 30. An even dividable cell culturing surface 30 may also be achieved even if the plug height is not equal to the top component thickness as shown in FIG. 4b. In such a case, the top component 10 may rest on the plugs 21 when the plugs 21 have entered the through holes 11 to thereby form open spaces between the bottom surface of the top component 10 and the top surface of the bottom component 20.

The embodiments are, however, not limited thereto. In other embodiments the thickness of the top component 10 may be larger than the height of the plugs 21 thereby forming respective wells when the plugs 21 have fully entered the through holes 11 as in the embodiments illustrated in FIGS. 3e and 3f.

In an embodiment, tops 24 of the multiple plugs 21 have a respective diameter or a diagonal within a range of 50 to 1000 μm, preferably within a range of 50 to 500 μm.

In an embodiment, a respective diameter or diagonal of tops 24 of the multiple plugs 21 is larger than half a respective height of the multiple plugs 21.

In a particular embodiment, a respective diameter or diagonal of tops 24 of the multiple plugs 21 is equal to or larger than a respective height of the multiple plugs 21.

In an embodiment, a respective top surface 25 of the multiple plugs 21 and/or a top surface 15 of the top component 10 are coated with a thermoresponsive polymer or a polymer mixture comprising the thermoresponsive polymer. The thermoresponsive polymer is preferably hydrophobic over a lower critical solution temperature (LCST) and is hydrophilic below the LCST.

In an embodiment, the top component 10 and the multiple plugs 21 are selected among polymer materials that can be coated by a cell adhesion promoting molecule, preferably selected from a group consisting of poly-D-lysine, poly-L-lysine, collagen, laminin and an extracellular matrix component.

In an embodiment, the cell culture assembly 1 also comprises a top frame 40 extending around a circumference of the top component 10 and being attached to the top component 10. The cell culture assembly 1 further comprises a bottom frame 50 extending around a circumference of the bottom component 20 and being attached to the bottom component 20, see FIGS. 5 and 6b.

In a particular embodiment, the cell culture assembly 1 further comprises multiple guiding pins arranged on one of the top frame 40 and the bottom frame 50. The cell culture assembly 1 also comprises multiple matching pin holes present in the other of the top frame 40 and the bottom frame 50. Each pin hole is arranged to receive a respective guiding pin when the top frame 40 is arranged on the bottom frame 50.

FIG. 5 schematically illustrates such an embodiment with guiding pins (cone-shaped plugs) and matching pin holes (holes) on diagonally opposite corners of the bottom frame 50 and the top frame 40.

In a particular embodiment, the guiding pins have walls that are fully or partially angled or sloped. Optionally, the matching holes may have walls that are fully or partially angled or sloped. Such sloped walls facilitates introducing the guiding pins into the matching pin holes.

In another embodiment, the cell culture assembly 1 comprises a top frame 40 extending around a circumference of the top component 10 and being attached to the top component 10. In this embodiment, the bottom component 20 constitutes a surface of a culture dish 55 or a well surface of a multiwell plate 55, see FIG. 6a.

Instead of having the surface of the culture dish 55 or multiwell plate 55, the bottom component 20 may constitute a surface of a plate or slide, such as microscope slide, that can be arranged into the culture dish 55 or multiwell plate 55.

In a particular embodiment, the cell culture assembly 1 further comprises multiple guiding pins arranged on one of the top frame 40 and at least one wall of the culture dish 55 or the multiwell plate 55. The cell culture assembly 1 also comprises multiple matching pin holes present in the other of the top frame 40 and the at least one wall of the culture dish 55 or the multiwell plate 55. Each pin hole is arranged to receive a respective guiding pin when the top frame 40 is arranged on the at least one wall of the culture dish 55 or the multiwell plate 55.

In a particular embodiment, the guiding pins have walls that are fully or partially angled or sloped. Optionally, the matching holes may have walls that are fully or partially angled or sloped. Such sloped walls facilitates introducing the guiding pins into the matching pin holes.

In an embodiment, the top component 10 comprises multiple wells 16 separated by well walls 17, for instance as shown in FIGS. 10a and 10b. In this embodiment, each well 16 of the multiple wells 16 comprises at least one through hole 11 of the multiple through holes 11.

In an embodiment, the cell culture assembly 1 comprises multiple wells 65 held together by a frame structure 60, see for instance FIGS. 8 and 9a. In this embodiment, each well 65 comprises a well bottom 61 constituting a respective top component 10 comprising multiple through holes 11 distributed in the fixed pattern. The cell culture assembly 1 also comprises a bottom tray 70 comprising a tray bottom 71 having multiple bottom components 20 defined thereon and at least one tray wall 73 surrounding the multiple bottom components 20. In this embodiment, each respective bottom component 20 comprises multiple plugs 21 distributed in the fixed pattern on the tray bottom 71 and arranged to be aligned with the multiple through holes 11 of a well 65 of the multiple wells 65.

The bottom tray 70 may have a single tray wall 73 such as when the bottom tray 70 have a circular or elliptical shape, whereas it typically comprises four or more tray walls 73 for other non-circular/elliptical shapes.

In a particular embodiment, the cell culture assembly 1 comprises multiple internal tray walls 74 connected to the tray bottom 71 and enclosing each respective bottom component 20.

In a particular embodiment, the cell culture assembly 1 comprises multiple guiding pins 72 arranged on one of the frame structure 60 and the at least one tray wall 73, see FIGS. 8 and 9a. The cell culture assembly 1 also comprises multiple matching pin holes 62 present in the other of the frame structure 60 and the at least one tray wall 73. In this particular embodiment, each pin hole 62 is arranged to receive a respective guiding pin 72 when the frame structure 60 is arranged on the at least one tray wall 73.

In another particular embodiment, the cell culture assembly 1 comprises multiple guiding pins 72 having walls that are fully or partially angled or sloped and having a larger top diameter than the multiple plugs 21, see FIG. 9b. In this particular embodiment, the multiple guiding pins 72 are arranged on one of i) the tray bottom 71 and ii) the well bottoms 61 or walls 66 of the wells 65. The cell culture assembly 1 also comprises, see FIG. 9c, multiple matching pin holes 62 preferably having walls that are fully or partially angled or sloped and arranged on the other of i) the tray bottom 71 and ii) the well bottoms 61 or the walls 66 of the wells 65. In this particular embodiment, each pin hole 62 is arranged to receive a respective guiding pin 72 when the multiple well bottoms 61 are arranged on the tray bottom 71.

Another aspect of the embodiments relates to a cell passaging method. The method comprises culturing (see FIG. 2a) cells 31 on a cell culturing surface 30 formed by a top surface 15 of a top component 10 of a cell culture assembly 1 according to the embodiments and respective top surfaces 25 of plugs 21 of a bottom component 20 of the cell culture assembly 1. The method also comprises removing (see FIG. 2c) the top component 10 from the bottom component 20. The method further comprises performing (see FIG. 2d) at least one of:

attaching the top component 10 onto a new bottom component 20′ lacking any cells 31 on respective top surfaces 25 of plugs 21 of the new bottom component 20′; and

attaching a new top component 10′ onto the bottom component 20. The new top component 10′ lacks any cells 31 on a top surface 15 of the new top component 10′.

Yet another aspect of the embodiments relates to a cell co-culturing method comprising culturing cells 31 on a cell culturing surface 30 formed by a top surface 15 of a top component 10 of a cell culture assembly 1 according to the embodiments and respective top surfaces 25 of plugs 21 of a bottom component 20 of the cell culture assembly 1. The method also comprises removing the top component 10 from the bottom component 20. The method further comprises performing at least one of:

attaching the top component 10 onto a new bottom component 20′ comprising cells 31 on respective top surfaces 25 of plugs 21 of the new bottom component 20′; and

attaching a new top component 10′ onto the bottom component 20. The new top component 10′ comprises cells 31 on a top surface 15 of the new top component 10′.

In an embodiment, culturing cells comprises culturing cells 31 of a first cell type on a cell culturing surface 30 of a first cell culture assembly 1 and culturing cells 31 of a second cell type on a cell culturing surface 30 of a second cell culture assembly 1. In such a case, the top component 10 of the first cell culture assembly 1 could be attached onto the bottom component 20 of the second cell culture assembly 1 and/or the top component 10 of the second cell culture assembly 1 could be attached onto the bottom component 20 of the first cell culture assembly 1.

The cell co-culturing method thereby enables co-culturing of different cell types in order to investigate the contact or interaction between the two cell types. This concept can of course be extended further by using, for instance, three different cell culture assemblies 1 and three different cell types.

A further aspect of the embodiments relates to a cell culture assembly 1 comprising a top component 10 comprising a cell culturing surface 15 and comprising multiple through holes 11 having at least one respective wall 12. The cell culture assembly 1 also comprises a bottom component 20 comprising multiple plugs 21 arranged to be aligned with the multiple through holes 11. Each plug 21 of the multiple plugs 21 comprises a cell culturing surface 25 and at least one wall 22. The cell culturing surface 15 of the top component 10 and the cell culturing surfaces 25 of the multiple plugs 21 form a dividable cell culturing surface 30 when the multiple plugs 21 are inserted in the multiple through holes 11. According to the embodiments, at least a bottom part or section 13 of the at least one wall 12 of the multiple through holes 11 is sloped or angled relative to a normal of the cell culturing surface 15 of the top component 10. In addition, or alternatively, at least a top part or section 24 of the least one wall 22 of the multiple plugs 21 is sloped or angled relative to a respective normal of the cell culturing surfaces 25 of the multiple plugs 21. The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

Culturing of HeLa and MCF-7 cells on uncoated and Poly-D-Lysine (PDL)-coated Polyethylene naphthalate (PEN) membranes, and testing the cells ability to cross from one membrane to another. The PEN membranes used in this study were prepared using commercially available PEN membrane slides with steel frames, commonly used for Laser Capture Microdissection (LCM).

Materials and Methods
HELA Cells, DMSZ ACC 57
MCF-7 Cells, DMSZ ACC 115
FBS Heat Inactivated. S.American500ML(ExpP60), Fisher Scientific 11550356
RPMI 1640 500ML, Fisher Scientific 10379144
Insulin, Recombinant Human SML, Fisher Scientific 11508856
Sodium Pyruvate MEM (100 mM), Fisher Scientific 11530396
MEM Non Essential Amino Acids (NEAA) (100×), liquid, Fisher Scientific 11350912
Penicillin-Streptomycin (Pen-Strep) Solution with 10,000 units Penicillin (Base)/ml and 10,000 μg Streptomycin (Base)/ML in 0.85% Saline, Fisher Scientific 11548876
Trypsin, 0.05% (1×) with EDTA 4Na, Fisher Scientific 11580626
Poly-D-Lysine 1 mg/ml, 20 ML, Fisher Scientific 11336634
PBS, DULBECCOS, Fisher Scientific 11590476
PEN Membrane frame slide, Life Technologies Europe BV LCM0521
Scalpels sterile No. 10 stainless-steel blade EA=10 PCS, Fisher Scientific 11342844
Plastic Petri Dishes, sterile 90MM100/PCS, Fisher Scientific 11551764
Plastic tweezers, sterile 128MM 50/PCS, Fisher Scientific 11336634

Coating Procedure

Membrane slides were coated by incubating with 1 ml Poly-D-Lysine diluted in sterilized water at a concentration of 50 μg/ml for 1 hour at 37° C. They were washed with sterilized water and air-dried for at least 2 hours.

UV Treatment

Coated and uncoated slides were exposed to irradiation with UV light at 254 nm for 30 minutes in a cell culture hood, in order to overcome the hydrophobic nature of the membrane, to sterilize and to destruct potentially contaminating nucleic acids.

Cell Preparation

HeLa cells were cultured in RPMI 1640, 10% heat inactivated FBS, 1% v/v Pen/Strep. MCF-7 cells were cultured in RPMI 1640, 10% heat inactivated FBS, 1×MEM non-essential amino acids, 1 mM sodium pyruvate, 10 μg/mL human insulin, 1% v/v Pen/Strep.

Before the experiment, both cell lines were passaged and fed twice weekly. At the day of the experiment, cell culture medium was removed and any residual medium was eliminated by rinsing the flasks with 6 ml of sterile DPBS. 1 ml of Trypsin-EDTA solution was added slowly to each flask and swirled to cover the cell monolayer. Flasks were incubated at 37° C. for 4 minutes. Cell detachment was controlled under microscope. The trypsin was inactivated by addition of 9 ml medium in each flask and the collected cell suspension was centrifuged at 1000 rpm for 4 minutes. The pellet was dissolved in 10 ml pre-warmed medium. Cells were counted, and HeLa and MCF-7 cells were diluted to cell concentrations of 0.15×10⁶ cells/mL and 0.25×10⁶ cells/mL, respectively.

Adding Cells to Membranes

UV-treated coated or uncoated membrane slides were placed in individual Petri dishes. 1 ml of cell suspension was added to each membrane. The membranes were incubated for 4 hours in 37° C. to allow attachment. Then 8 mL cell culture medium was pipetted gently into each Petri dish, outside the membrane slide. The membranes were incubated overnight at 37° C., 5% CO₂. Each cell line was tested with both coated and uncoated membranes, and every experiment condition was performed in triplicate.

Membrane Manipulation and Testing

Membranes were cut using scalpels. One half of the membrane was cut out from each membrane slide with cells attached. A new UV-treated membrane slide without cells, coated or uncoated, was placed under the membrane slide with cells, so that the two membranes were in direct contact with each other; that is, on the bottom slide the metal frame was under the membrane, and on the top slide the metal frame was situated on top of the membrane (any side of the membrane could be used for culture). This made it possible for the cells to cross over to the new membrane in the area that was cut out from the membrane with cells. The dishes with membrane slides and cells were returned to the incubator, and the status of the cells was checked and pictures were taken at different days.

Results

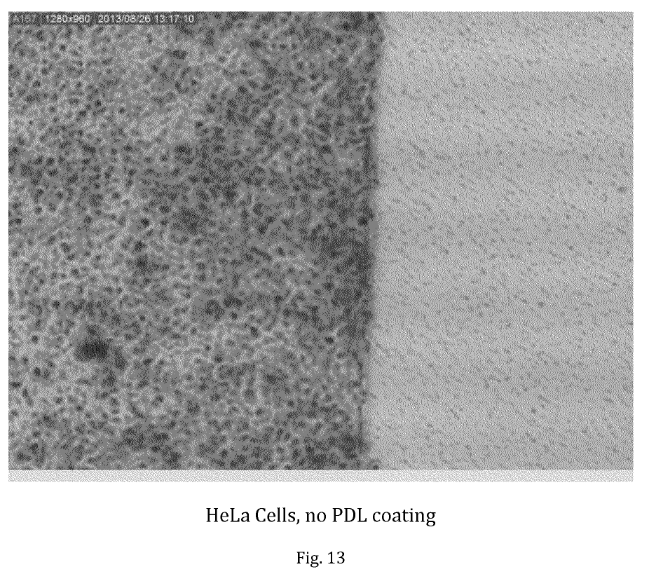

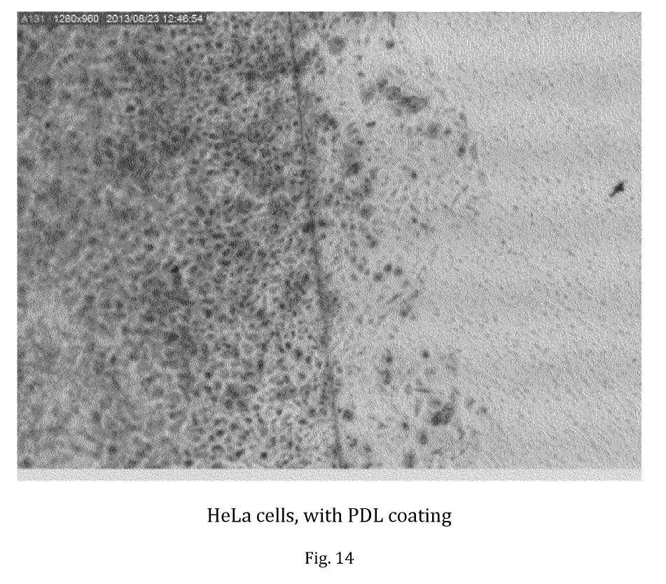

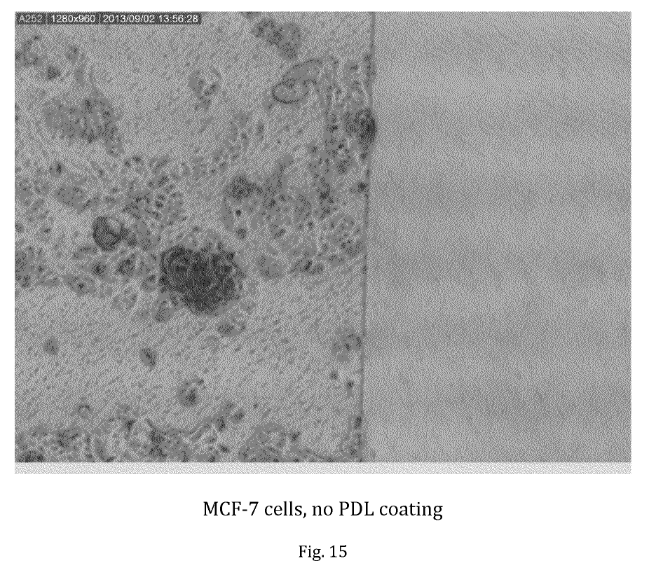

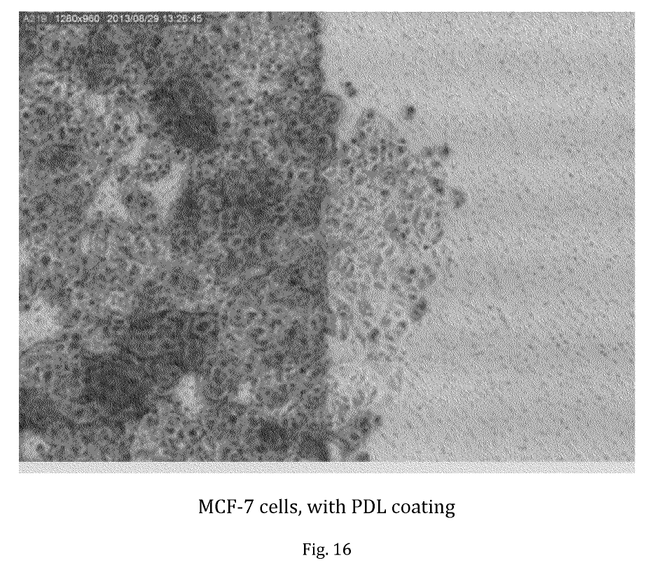

Neither of the cell lines could grow over to uncoated membranes (FIGS. 13 and 15). In contrast, both cell lines were shown to grow over to the bottom membrane if the membranes were coated with Poly-D-Lysine (FIGS. 14 and 16). Cells did not cross the entire edge between membranes, probably due to the fact that the membranes had a tendency to become “wavy” after cutting with a scalpel.

### Example 2

Cell culture assemblies such as those shown in FIGS. 5, 7, 8 and 9 could be modeled using CAD software and manufactured using 3D printing. Such cell culture assemblies could be sterilized by UV light as described in Example 1, or by immersing the device in 70% ethanol for 15 minutes and then allowing it to dry in a sterile cell culture hood, or a combination of both methods. The cell culture assembly shown in FIG. 5 could be placed in a standard culture dish and immersed in culture medium. The holding frame shown in FIG. 7 could be combined with a second holding frame, as illustrated in FIG. 6b, and used with standard multiwell plates, or combined with a multiwell plate where plugs are integrated in the bottom as shown in FIG. 6a. The cell culture assemblies shown in FIGS. 8 and 9 could be free-standing devices with culture medium added to the wells by standard pipetting.

Cells, such as HeLa cells, could be prepared as described in Example 1 and cultured with medium as described in Example 1. The cells could then be added by standard pipetting and incubated in 37° C., 5% CO₂. When components are exchanged during cell passaging, or cell splitting, the cell culture assembly shown in FIG. 5 could, for example, be manipulated with forceps, while cell culture assemblies such as those shown in FIGS. 7, 8 and 9 could be manipulated by hand or by a robotic device. Culture medium could further be replaced with new medium before or after the exchange of assembly components.

### REFERENCES

Errando-Herranz C, Vastesson A, Zelenina M. Biocompatibility of OSTE polymers studied by cell growth experiments. In Proceedings of the 17th Int. Conf. on Miniaturized Systems for Chemistry and Life Sciences (microTAS): (2013)
Haraguchi Y, Shimizu T, Sasagawa T, Sekine H, Sakaguchi K, Kikuchi T, Sekine W, Sekiya S, Yamato M, Umezu M, Okano T. Fabrication of functional three-dimensional tissues by stacking cell sheets in vitro. Nat Protoc. 2012 Apr. 5; 7(5):850-8
Hohenstein Elliott K A, Peterson C, Soundararajan A, Kan N, Nelson B, Spiering S, Mercola M, Bright GR. Laser-Based Propagation of Human iPS and ES Cells Generates Reproducible Cultures with Enhanced Differentiation Potential. Stem Cells Int. 2012; 2012:926463.
Majd H, Wipff P J, Buscemi L, Bueno M, Vonwil D, Quinn T M, Hinz B. A novelmethod of dynamic culture surface expansion improves mesenchymal stem cell proliferation and phenotype. Stem Cells. 2009 January; 27(1):200-9.
Mitalipova M M, Rao R R, Hoyer D M, Johnson J A, Meisner L F, Jones K L, Dalton S, Stice S L. Preserving the genetic integrity of human embryonic stem cells. Nat Biotechnol. 2005 January; 23(1):19-20.
Muraoka M, Shimizu T, Itoga K, Takahashi H, Okano T. Control of the formation of vascular networks in 3D tissue engineered constructs. Biomaterials. 2013 January; 34(3):696-703
Nagase K, Kobayashi J, Okano T. Temperature-responsive intelligent interfaces for biomolecular separation and cell sheet engineering. J R Soc Interface. 2009 Jun. 6; 6 Suppl 3:S293-309
Vancha A R, Govindaraju S, Parsa K V, Jasti M, Gonzâlez-Garcîa M, Ballestero R P. Use of polyethyleneimine polymer in cell culture as attachment factor and lipofection enhancer. BMC Biotechnol. 2004 Oct. 15; 4:23

1. A cell culture assembly comprising:
a top component comprising multiple through holes distributed in a fixed pattern; and
a bottom component comprising multiple plugs distributed in said fixed pattern and arranged to be aligned with said multiple through holes,
wherein said multiple plugs fit in said multiple through holes, wherein said multiple through holes comprise walls that are fully or partially angled or sloped and/or said multiple plugs comprise walls that are fully or partially angled or sloped, and
wherein a respective base of said multiple through holes is wider than a respective top of said multiple through holes and/or a respective base of said multiple plugs is wider than a respective top of said multiple plugs.

2. (canceled)

3. The cell culture assembly according to claim 1, wherein said multiple through holes have a round shape and said walls of said multiple through holes are chamfered or beveled to have a larger diameter at said respective base of said multiple through holes as compared to at said respective top of said multiple through holes; and/or
said multiple plugs have a shape of a respective chamfered circular cylinder or truncated circular cone and have a larger diameter at said respective base of said multiple plugs as compared to at said respective top of said multiple plugs.

4.-5. (canceled)

6. The cell culture assembly according to claim 1, wherein said walls of said multiple through holes are vertical; and
said walls of said multiple plugs are fully or partially angled or sloped thereby forming a gap between a respective top of said multiple plugs and a respective top of said walls of said multiple through holes.

7. The cell culture assembly according to claim 1, wherein top surfaces of said multiple plugs and a top surface of said top component collectively form a dividable cell culturing surface.

8. The cell culture assembly according to claim 7, wherein said top surfaces of said multiple plugs and said top surface of said top component form an essentially aligned and even dividable cell culturing surface.

9. The cell culture assembly according to claim 1, wherein tops of said multiple plugs have a respective diameter or a diagonal within a range of 50 to 1000 μm, optionally wherein the respective diameter or diagonal is within a range of 50 to 500 μm.

10. The cell culture assembly according to claim 1, wherein a respective diameter or diagonal of said tops of said multiple plugs is equal to or larger than a respective height of said multiple plugs.

11. The cell culture assembly according to claim 1, wherein a respective top surface of said multiple plugs and/or a top surface of said top component are coated with a thermoresponsive polymer or a polymer mixture comprising said thermoresponsive polymer; and
said thermoresponsive polymer is hydrophobic over a lower critical solution temperature (LCST) and is hydrophilic below said LCST.

12. The cell culture assembly according to claim 1, wherein said top component and said multiple plugs comprise polymer materials that can be coated by a cell adhesion promoting molecule, optionally wherein the polymer materials are selected from a group consisting of poly-D-lysine, poly-L-lysine, collagen, laminin and an extracellular matrix component.

13. The cell culture assembly according to claim 1, further comprising a top frame extending around a circumference of said top component and attached to said top component; and
a bottom frame extending around a circumference of said bottom component and attached to said bottom component.

14. The cell culture assembly according to claim 13, further comprising multiple guiding pins arranged on one of said top frame and said bottom frame; and
multiple matching pin holes present in the other of said top frame and said bottom frame, wherein each pin hole of said multiple matching pin holes is arranged to receive a respective guiding pin of said multiple guiding pins when said top frame is arranged on said bottom frame.

15. The cell culture assembly according to claim 1, further comprising a top frame extending around a circumference of said top component and being attached to said top component, wherein said bottom component constitutes a surface of a culture dish or a well surface of a multiwell plate.

16. The cell culture assembly according to claim 15, further comprising multiple guiding pins arranged on one of said top frame and at least one wall of said culture dish or said multiwell plate; and
multiple matching pin holes present in the other of said top frame and said at least one wall of said culture dish or said multiwell plate, wherein each pin hole of said multiple matching pin holes is arranged to receive a respective guiding pin of said multiple guiding pins when said top frame is arranged on said at least one wall of said culture dish or said multiwell plate.

17. The cell culture assembly according to claim 1, wherein said top component further comprises multiple wells separated by well walls and each well of said multiple wells comprises at least one through hole of said multiple through holes.

18. The cell culture assembly according to claim 1, further comprising multiple wells held together by a frame structure, wherein each well comprises a well bottom constituting said top component comprising said multiple through holes distributed in said fixed pattern; and
a bottom tray comprising a tray bottom comprising a plurality of said bottom components defined thereon and at least one tray wall surrounding said plurality of said bottom components, wherein each respective bottom component of said plurality of said bottom components comprises said multiple plugs distributed in said fixed pattern on said tray bottom and arranged to be aligned with said multiple through holes of a well of said multiple wells.

19. The cell culture assembly according to claim 18, further comprising multiple internal tray walls connected to said tray bottom and enclosing each respective bottom component.

20. The cell culture assembly according to claim 18, further comprising multiple guiding pins arranged on one of said frame structure and said at least one tray wall; and
multiple matching pin holes present in the other of said frame structure and said at least one tray wall, wherein each pin hole of said multiple matching pin holes is arranged to receive a respective guiding pin of said multiple guiding pins when said frame structure is arranged on said at least one tray wall.

21. The cell culture assembly according to claim 18, further comprising multiple guiding pins having walls that are fully or partially angled or sloped and having a larger top diameter than said multiple plugs, wherein said multiple guiding pins are arranged on one of i) said tray bottom and ii) said well bottoms (61) or walls of said wells; and
multiple matching pin holes arranged on the other of i) said tray bottom and ii) said well bottoms or said walls of said wells, wherein each pin hole of said multiple matching pin holes is arranged to receive a respective guiding pin of said multiple guiding pins when said multiple well bottoms are arranged on said tray bottom, and optionally wherein walls of said multiple matching pin holes are fully or partially angled or sloped.

22. A cell passaging method comprising:
culturing cells on a cell culturing surface formed by a top surface of said top component of said cell culture assembly according to claim 1 and respective top surfaces of said multiple plugs of said bottom component of said cell culture assembly;
removing said top component from said bottom component; and
performing at least one of:
attaching said top component onto a new bottom component comprising multiple plugs lacking any cells on respective top surfaces of said multiple plugs of said new bottom component; and
attaching a new top component onto said new bottom component, said new top component lacking any cells on a top surface of said new top component.

23. A cell co-culturing method comprising:
culturing cells on a cell culturing surface formed by a top surface of said top component of said cell culture assembly according to claim 1 and respective top surfaces of said multiple plugs of said bottom component of said cell culture assembly;
removing said top component from said bottom component; and
performing at least one of:
attaching said top component onto a new bottom component comprising cells on respective top surfaces of said multiple plugs of said new bottom component; and
attaching a new top component onto said new bottom component, said new top component comprising cells on a top surface of said new top component.

24. A cell culture assembly comprising:
a top component comprising a cell culturing surface and comprising multiple through holes having at least one respective wall;
a bottom component comprising multiple plugs arranged to be aligned with said multiple through holes, wherein each plug of said multiple plugs comprises a cell culturing surface and at least one wall, wherein said cell culturing surface of said top component and said cell culturing surfaces of said multiple plugs form a dividable cell culturing surface when said multiple plugs are inserted in said multiple through holes; and wherein at least a bottom part of said at least one wall of said multiple through holes is sloped or angled relative to a normal of said cell culturing surface of said top component; and/or
at least a top part of said at least one wall of said multiple plugs is sloped or angled relative to a respective normal of said cell culturing surfaces of said multiple plugs.
